(19) United States
(12) Patent Application Publication
(10) Pub. No.: US 2024/0242801 A1
(43) Pub. Date: Jul. 18, 2024

(54) SYSTEMS AND METHODS FOR PATIENT HISTORY ANALYSIS AND TIME-LINE SEGMENTATION

(71) Applicant: GE Precision Healthcare LLC, Waukesha, WI (US)

(72) Inventors: Sanand Sasidharan, Bangalore (IN); Anuradha Anuradha, Bangalore (IN); Pradeep Pamidi, Bangalore (IN)

(21) Appl. No.: 18/559,291

(22) PCT Filed: Dec. 21, 2021
(86) PCT No.: PCT/US2021/064723

§ 371 Date: Nov. 6, 2023

(30) Foreign Application Priority Data
May 31, 2021 (IN) 202141024146

(51) Int. Cl. G16H 15/00; G16H 10/60

(52) U.S. Cl. CPC G16H 15/00; G16H 10/60

(57) ABSTRACT

Systems and methods for patient history analysis and time-line segmentation are provided. The method comprises acquiring health records of a patient using a plurality of medical devices over a duration of treatment. The health records comprise at least one action or an event related to the patient and storing the health records in electronic format. The method further comprises identifying beginning and end of at least one action or the event related to the patient using a begin-end marker detection module and mark the beginning and end boundary of at least one action or the event. The method further comprises segmenting the at least one action or the event by generating ambiguous timeline boundaries of the at least one action or the event using a segment begin-end detection module. The method further comprises demarcating the segment boundaries and arranging the segments in order of timeline.

### CROSS-REFERENCE TO RELATED APPLICATIONS

This application is a U.S. national stage entry under 35 U.S.C. § 371 to Patent Cooperation Treaty application number PCT/US2021/064723 entitled “SYSTEMS AND METHODS FOR PATIENT HISTORY ANALYSIS AND TIME-LINE SEGMENTATION” filed Dec. 21, 2021 which claims priority to India filing 202141024146 entitled “SYSTEMS AND METHODS FOR PATIENT HISTORY ANALYSIS AND TIME-LINE SEGMENTATION” filed May 31, 2021. These disclosures are hereby incorporated by reference in their entirety.

### TECHNICAL FIELD

This application relates to analysis of the patient information, and more particularly to systems and methods for patient history analysis and time-line segmentation.

### BACKGROUND

Hospitals, doctors and paramedical staff are increasingly relying on digitally obtaining, processing, storing and retrieving the medical records of a subject patient. These medical records that are generated during investigations of the subject include a variety of examinations like blood tests, urine tests, pathology reports, scanning using various medical imaging systems like ultrasound, magnetic resonance imaging, computed tomography systems and other radiological investigations. These medical records generated from various investigations may be stored in different formats, for example, the medical images will be stored in a format popularly known as DICOM that is different than the format for storing the blood parameter reports which is still different than the format of storing the pathological reports. For a large hospital facility, retrieving all the relevant information stored in different formats and presenting it to a medical practitioner for analysis is challenging.

Duration of the diagnosis of a medical condition of a subject followed by treatment may be spread over time from few days to few months or even years in case of chronic diseases. Diseases that take more than one year to cure are considered chronic diseases. Some of the well-known chronic diseases may include for example Alzheimer's disease, Cancer, Asthma, Diabetes, and others. It may take long before the actual disease or medical condition of the subject is accurately diagnosed, and correct course of treatment begins. In one example, a patient may ignore his mild headache or may be given less sophisticated medication that temporarily relieves headache for few months. Later as the condition of the patient starts worsening or getting complicated, more investigations may be carried out including for example magnetic resonance imaging of the brain that may reveal multiple blockages of the arteries or presence of a tumor. At the same time, blood investigations may reveal diabetic condition and the medical practitioner may start initial preventive treatment to minimize complications. Further the medical practitioner may decide to carry out a biopsy to check for presence of cancer cells in the tumor and the further treatment may vary based on the pathological reports of the biopsy. When presence of cancerous cells is detected, the doctor will determine the stage of the cancer and follow treatment protocols for the stage of cancer. Different available treatments including surgery, chemotherapy, radiotherapy may be provided to the patient based on the stage of the cancer. Further, medications may be altered based on the assessed parameters of the patient body such as various blood parameter counts, urine reports and other examinations. The patient may move from one hospital to another or from one country to another for treatment and keeping the timely records of the medical condition, symptoms and diagnosis, and the treatment offered during particular condition is challenging.

Further, some of these records may be in non-digital format, for example the doctor's prescription for the initial headache or the tablets and dosages suggested for diabetes may be on a paper prescription. After few months or years of treatment, the documents may be lost or the conditions under which the medication was offered may not be retrievable. When the medical practitioner reviews the records after few months or years, it may be difficult for him to go through all the records in a sequential manner and understand the history of the patient in a detail. Critical facts or details may be missed while the doctor goes through the records.

Doctors are increasingly relying on Electronic Health Record (EHR) systems to go through historical health records of the patient during diagnosis. For patients with chronic illnesses, there are often hundreds of EHRs from many routine visits. Going through past EHRs for such patients is a slow process and there is a chance of missing important records which are spread out between large number of less informative routine visit records. This in turn has a negative impact on the time spend talking to the patient and on the accuracy of diagnosis and quality of treatment.

Systems and methods are required for storing and segmenting the electronic health records and presenting the health records in sync with the medical condition of the patient to more clearly demarcate only relevant EHRs.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments described herein, systems, computer-implemented methods, apparatus and/or computer program products are described that facilitate domain adaptation of image processing models.

In accordance with an aspect of the disclosure a method is disclosed. The method comprises acquiring health records of a patient using a plurality of medical devices over duration of a treatment, wherein the health records comprise at least one action or an event related to the patient. The method further comprises identifying beginning and end of at least one action or the event related to the patient and marking the beginning and end boundary of at least one action or the event. The method further comprises segmenting the at least one action or the event by generating ambiguous timeline boundaries of the at least one action or the event. The method further comprises demarcating the segment boundaries and arranging the segments in order of timeline.

In accordance with an aspect of the disclosure a system is disclosed. The system for patient history analysis and time-line segmentation comprises a hearth record system configured to store a plurality of health records of a patient acquired over duration of a treatment. The system further comprises a begin-end marker detection module connected to the health record system to retrieve the health records of the patient, wherein the begin-end marker detection module is configured to identify the beginning and end of at least one action or the event related to the patient and mark the beginning and end boundary of the at least one action or the event. The system further comprises a segment begin-end detection module configured to segment the at least one action, or the event related to the patient by generating ambiguous timeline boundaries of the at least one action or the event. The system further comprises a gap handling configured to demarcate the segment boundaries and a hierarchy module configured arrange the segments in order of timeline.

In some embodiments, elements described in the disclosed systems can be embodied in different forms such as a computer-implemented method, a computer program product, or another form.

### DESCRIPTION OF THE DRAWINGS

FIG. 1(a) illustrates the existing system for displaying the clinical information of a patient to a user in accordance with an aspect of the disclosure.

FIG. 1(b) illustrates a system for displaying the clinical information of a subject patient to a user in accordance with an aspect of the disclosure.

FIG. 2 illustrates a system for patient history analysis and time-line segmentation using an electronic health record display system in accordance with an aspect of the disclosure.

FIG. 3 illustrates a method for timeline segmentation and display of the electronic health records of the patient over the display device in accordance with an aspect of the disclosure.

FIG. 4 illustrates a method for timeline segmentation and display of the electronic health records of the patient over the display device in accordance with an aspect of the disclosure.

FIG. 5 illustrates a method for timeline segmentation and display of the electronic health records of the patient over the display device in accordance with an aspect of the disclosure

### DETAILED DESCRIPTION

The following detailed description is merely illustrative and is not intended to limit embodiments and/or application or uses of embodiments. Furthermore, there is no intention to be bound by any expressed or implied information presented in the preceding Summary section or in the Detailed Description section.

In the following specification and the claims, reference will be made to a number of terms, which shall be defined to have the following meanings.

The singular forms “a”, “an”, and “the” include plural references unless the context clearly dictates otherwise.

As used herein, the terms “software” and “firmware” are interchangeable and include any computer program stored in memory for execution by devices that include, without limitation, mobile devices, clusters, personal computers, workstations, clients, and servers.

As used herein, the term “computer” and related terms, e.g., “computing device”, “computer system” “processor”, “controller” are not limited to integrated circuits referred to in the art as a computer, but broadly refers to at least one microcontroller, microcomputer, programmable logic controller (PLC), application specific integrated circuit, and other programmable circuits, and these terms are used interchangeably herein.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

As used herein, the terms “systems”, “devices” and “apparatuses are interchangeable and include components, sub-components, sub-systems that include without limitation the medical imaging devices.

In accordance with an aspect of the disclosure a method is disclosed. The method comprises acquiring health records of a patient using a plurality of medical devices over duration of a treatment, wherein the health records comprise at least one action or an event related to the patient. The method further comprises identifying beginning and end of at least one action or the event related to the patient and marking the beginning and end boundary of at least one action or the event. The method further comprises segmenting the at least one action or the event by generating ambiguous timeline boundaries of the at least one action or the event. The method further comprises demarcating the segment boundaries and arranging the segments in order of timeline.

In accordance with an aspect of the disclosure a system is disclosed. The system for patient history analysis and time-line segmentation comprises a hearth record system configured to store a plurality of health records of a patient acquired over duration of a treatment. The system further comprises a begin-end marker detection module connected to the health record system to retrieve the health records of the patient, wherein the begin-end marker detection module is configured to identify the beginning and end of at least one action or the event related to the patient and mark the beginning and end boundary of the at least one action or the event. The system further comprises a segment begin-end detection module configured to segment the at least one action, or the event related to the patient by generating ambiguous timeline boundaries of the at least one action or the event. The system further comprises a gap handling configured to demarcate the segment boundaries and a hierarchy module configured arrange the segments in order of timeline.

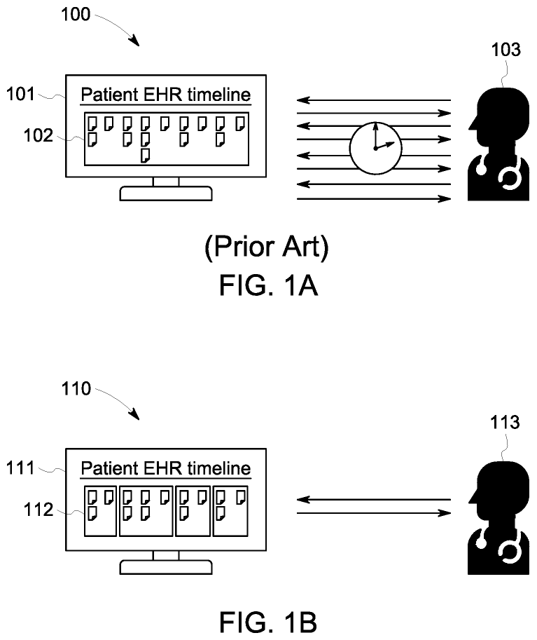

Embodiments of the present disclosure will now be described, by way of example, with reference to the figures, in which FIG. 1(a) describes the existing system (100) for displaying the clinical information (102) of a subject patient to a user (103). The display (101) may be any known device having a screen to display the clinical information (102). The clinical information (102) as shown in FIG. 1(a) may have been acquired over longer duration of time exceeding one year and stored in the memory of a computer system (not shown). This clinical information (102) may be generated during investigations of the subject patient and the clinical information (102) may include a variety of examination reports, for example blood tests, urine tests, pathology reports, scanning using various medical imaging systems like ultrasound, magnetic resonance imaging, computed tomography systems and other radiological investigations. These medical records generated from various investigations may be stored in different formats, for example, the medical images will be stored in a digital imaging in medical communication (DICOM) format that is different than the format for storing the blood parameter reports which is still different than the format of storing the pathological reports. For a large hospital facility, retrieving all the relevant clinical information (102) stored in different formats and presenting the clinically significant portions of the information (102) to the user (103) for analysis is challenging.

FIG. 1(b) shows a system (110) according to an aspect of the disclosure. The system (110) according to an aspect of the disclosure may be used for displaying the clinical information (112) of a subject patient to a user (113). The display (111) may be any known device having a screen to display the clinical information (112). The clinical information (112) as shown in FIG. 1(b) may have been acquired over time and stored in the memory of a computer system (not shown). This clinical information (112) may be generated during investigations of the subject patient and the clinical information (112) may include a variety of examination reports for example blood tests, urine tests, pathology reports, scanning using various medical imaging systems like ultrasound, magnetic resonance imaging, computed tomography systems and other radiological investigations. In accordance with an aspect of the disclosure, the clinical information (112) may be partitioned or segmented based on the patient's clinical history timeline into subsections 112 (a), 112 (b), and 112 (c) based on patient's condition or treatment type. Although only three subsections 112 (a), 112 (b), and 112 (c) of the clinical information (112) have been mentioned, it is apparent that more than three subsections of the information are possible. The clinical information (112) may be arranged to indicate the important events, decisions and highlight only those features that are clinically relevant for the user. Further the clinical information (112) may be segregated to in a variety of user-friendly display formats, for example, bars indicating beginning of the particular treatment and end of the treatment. When different treatments were offered simultaneously, the display will show two different bars over a timeline with overlapping portions.

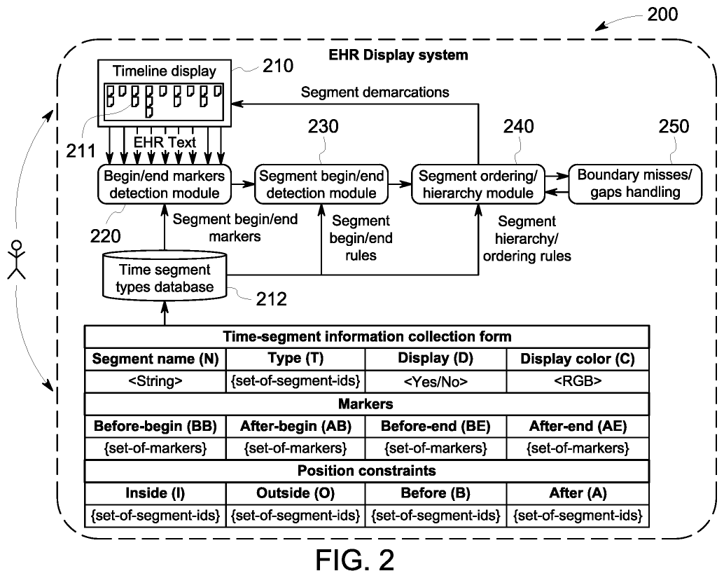

In accordance with an aspect of the disclosure, FIG. 2 shows a system (200) for patient history analysis and time-line segmentation using an electronic health record display system. The system (200) may include a display (210) configured to show the electronic health records (211) of the patient according to an aspect of the disclosure. The electronic health records of the patient may be stored in a memory of a computer system (212) or a server or any other electronic format known in the art or developed in the future. The computer system may be connected to a begin-end marker detection module (220). The begin-end detection module (220) may be configured to identify the beginning and end of a particular action or an event related to the patient and mark the beginning and end boundary of the action or the event. In one non-limiting example, the beginning event or the action may be admission of the patient to the hospital for diagnosis. Further, the patient may have been subject to magnetic resonance imaging (MRI) of the skull region. If no tumor was seen in the initial MR images, the patient may be discharged at a later point of time. This marks the end of imaging action and discharge of the patient. This scanning information of the patient in past may be time stamped and stored in the computer memory or any other suitable electronic storage medium. The begin-end marker detection module (220) may be configured to access the scanning information from the electronic storage medium. From the information stored on the electronic storage medium, the begin-end marker detection module (220) may be further configured to identify the scanning timeline including the date of admission of the patient, duration of the examination and display the date of discharge of the patient with a major conclusion that no tumor was initially detected. The begin-end marker detection module (220) may be configured to identify all such actions or events associated with the patient over the entire duration of the treatment. In one aspect of the disclosure, the begin-end marker detection module (220) may be configured to read the text information stored in the electronic storage medium and convert the information into a user viewable graph.

In accordance with an aspect of the disclosure the system (200) further comprises a segment begin-end detection module (230). The segment begin-end detection module (230) may be configured to segment the various actions or events identified by the begin-end marker detection module (220). Segmenting the actions or the events identified by the begin-end marker detection module (220) by the segment begin-end detection module (230) comprises identifying the timeline boundaries of the actions or the events and arranging the segmented actions or the events according to the timeline of the action or the event. In one non-limiting example, the patient may have undergone a treatment for various unrelated medical conditions like brain tumor, chest infection, spinal dislocation and liver cirrhosis. According to an aspect of the disclosure, the segment begin-end detection module (230) may be configured to segment all the actions taken for these medical conditions and show the corresponding medication offered for it. The segment begin-end detection module (230) correlates the medical condition with the various other parameters including for example the timeline of the medical condition, medication offered against the medical condition. The segment begin-end detection module (230) further creates a visual graph showing the timelines of medications along with the overlaps in the treatment timelines. However, if the specific data related to end of the treatment timeline is not available, or there are breaks and restarts in the treatment, the boundaries of the graph for such treatment become uncertain and may get mixed with the other graphs. In accordance with an aspect of the disclosure, a hierarchy module (240) may be provided. The hierarchy module (240) may be configured to arrange the segments in order of the timeline. The gaps or the indefinite boundaries of the segments need to be identified for sequencing the segments in order of timeline.

A gap handling module (250) may be connected to the hierarchy module to identify and mitigate the gaps between two segments along the timeline wherever the timeline data is available. Further, an option may be provided to a clinician to manually fill the data and update the timelines based on the available medical records. This minimizes the timeline gaps and provides more accurate picture to the user viewing the patient timeline. The demarcated segments may be sent to the display (210) by the hierarchy module (240) for showing the timeline sequence of the actions or the events related to the patient.

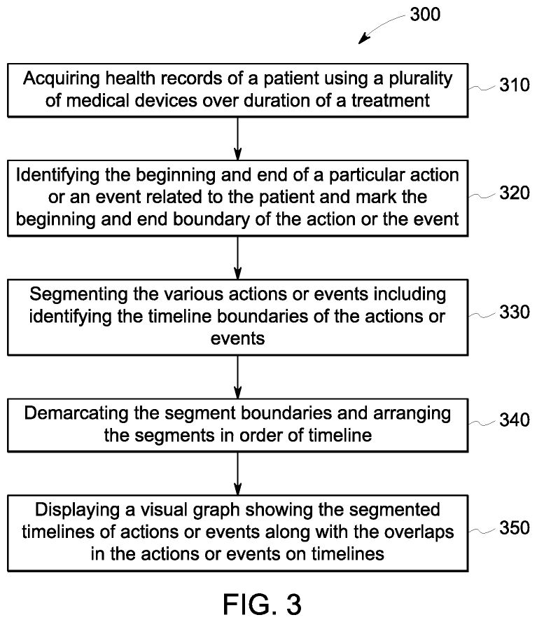

FIG. 3 shows a method (300) for patient history analysis and timeline segmentation of the electronic health records and displaying segmented health records of the patient over the display device. The method (300) comprises acquiring (310) health records of a patient over a period of time and store the records for future records. The health records may be electronic health records (EHRs) or physical health records that may be manually entered into the electronic health record systems. Although the reference is made to a single patient, it may be apparent to those skilled in the art that the health records of many such patients may be simultaneously acquired and stored. The electronic health records of the patient may be stored in a memory of a computer system (not shown) or a server or any other electronic format known in the art or developed in the future. The method (300) further comprises identifying (320) the beginning and end of a particular action or an event related to the patient and mark the beginning and end boundary of the action or the event. Identifying (320) the beginning and end of a particular action or an event may be carried out using a begin-end marker detection module (220). Various treatments of a patient may have begun at various times and continued for a specific duration before end of the treatment. The treatment course may include medication, visits to clinics, scanning, therapies, admission to the hospital, blood and other tests, discharge from the hospital and post discharge medication. The begin-end marker detection module (220) may be configured to access the scanning information from the electronic storage medium. From the information stored on the electronic storage medium, the begin-end marker detection module (220) may be further configured to identify the scanning timeline including the date of admission of the patient, duration of the examination and display the date of discharge of the patient with a major outcome or conclusion for example no serious ailment was initially detected. The begin-end marker detection module (220) may be configured to identify all such actions or events associated with the patient over the entire duration of the treatment.

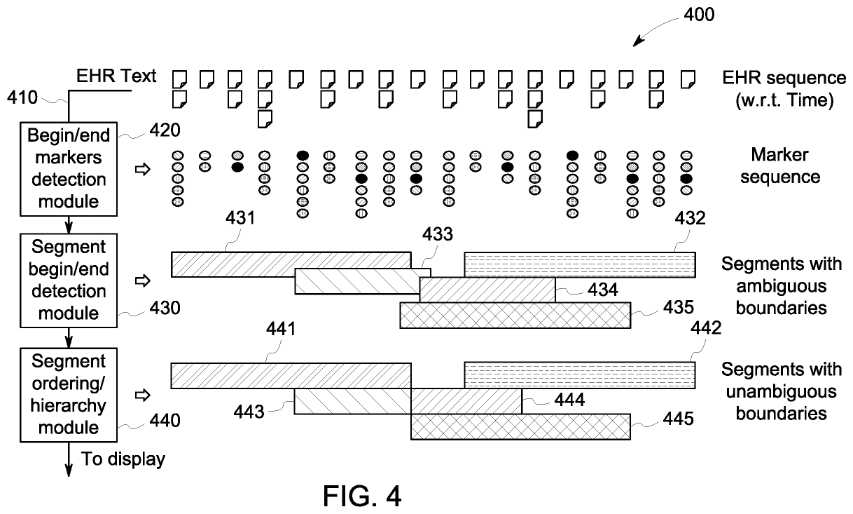

The method (300) further comprises segmenting (330) the various actions or events including identifying the timeline boundaries of the actions or the events. Segmenting (330) the various actions or the events may be carried out using a segment begin-end detection module (230). The segment begin-end detection module (230) may be configured to segment the various actions or the events identified by the begin-end marker detection module (220). Segmenting the actions or the events identified by the begin-end marker detection module (220) by the segment begin-end detection module (230) comprises identifying the timeline boundaries of the actions or events. In one non-limiting example, the patient may have undergone a treatment for various unrelated medical conditions like brain tumor, chest infection, spinal dislocation and liver cirrhosis. According to an aspect of the disclosure, segmenting (330) using the segment begin-end detection module (230) may be carried out to segment all the different actions taken for these medical conditions and show the corresponding medication offered for each medical condition. The segment begin-end detection module (230) correlates the medical condition with the various other parameters including for example the timeline of the medical condition and the medication offered against the medical condition. In instances where the end time of the action or the event is not properly identifiable, an ambiguous boundary may be generated. An ambiguous boundary is one where two segments overlap (shown in FIG. 4) and demarcation of the segments is not possible. If the specific data related to end of the action or event timeline is not available, or there are breaks and restarts in the treatment, the boundaries of the graph for such treatment become uncertain and may get mixed with the other graphs.

The method (300) further comprises demarcating (340) the segment boundaries and arranging the segments in order of timeline. Demarcating (340) the segment boundaries comprises separating the segments from one another and create identifiable boundaries. Arranging the segments in order of the timeline comprises identifying the sequence of the various actions or events related to a patient and arranging the sequence of actions or events along the timeline. In accordance with an aspect of the disclosure, a hierarchy module (240) may be provided. The hierarchy module (240) may be configured to arrange the segments in order of the timeline along with the clearly demarcated boundaries. Arranging the segments in sequential order or timeline creates the time sequence of the actions or events related to the patient. Arranging (340) the segments in order of the timeline may further comprise generating a visual graph showing the timelines of actions or events along with the overlaps in the action timelines.

The method (300) further comprises displaying (350) a visual graph showing the segmented timelines of actions or events containing the demarcated boundaries of the actions or events on timelines. The demarcated segments may be sent to the display device (210) by the hierarchy module (240) for showing the timeline sequence of the actions or the events related to the patient. The display (210) may be configured to show the timeline sequence of the actions or the events related to the patient along with the clinically important actions from every segment of the timeline. Displaying (350) may comprise showing the action or event segments in a variety of user-friendly display formats, for example, bars indicating beginning of the particular treatment and end of the treatment. When different treatments were offered simultaneously, the display will show two different segmented bars over a timeline with overlapping portions. The method (300) may be explained with an example of FIG. 4.

In accordance with an aspect of the disclosure, FIG. 4 shows an example method (400) for timeline segmentation and display of the electronic health records of the patient over the display device. The method (400) may include a display (not shown) configured to show the electronic health records (410) of the patient according to an aspect of the disclosure. The electronic health records (410) of the patient may be stored in a memory of a computer system (not shown) or a server or any other electronic format known in the art or developed in the future.

The computer system may be connected to a begin-end marker detection module (420). The begin-end detection module (420) may be configured to identify the beginning and end of a particular action or an event related to the patient and mark the beginning and end boundary of the action or the event. Various color schemes and shapes may be used to represent the type of disease, treatment offered and duration for a patient.

In one non-limiting example, the beginning event or the action may be admission of the patient to the hospital for diagnosis. A color-coded marker sequence may be generated corresponding to various electronic health records (EHRs) by the begin-end marker detection module. Further, the patient may have been subject to magnetic resonance imaging (MRI) for skull region. If no tumor was seen in the initial images, the patient may be discharged at a later point of time. This marks the end of imaging action and discharge of the patient. This scanning information of the patient in past may be time stamped and stored in the computer memory or any other suitable electronic storage medium. The begin-end marker detection module (420) may be configured to access the scanning information from the electronic storage medium. From the information stored on the electronic storage medium, the begin-end marker detection module (420) may be further configured to identify the scanning timeline including the date of admission of the patient, duration of the examination and display the date of discharge of the patient with a major conclusion that no tumor was initially detected. The begin-end marker detection module (420) may be configured to identify all such actions or events associated with the patient over the entire duration of the treatment. In one aspect of the disclosure, the begin-end marker detection module (420) may be configured to read the text information stored in the electronic storage medium and convert the information into a user viewable graph.

In accordance with an aspect of the disclosure the system (400) further comprises a segment begin-end detection module (430). The segment begin-end detection module (430) may be configured to segment the various actions or events identified by the begin-end marker detection module (420). Segmenting the actions or events identified by the begin-end marker detection module (420) by the segment begin-end detection module (430) comprises identifying the timeline boundaries of the actions or events and arranging the segmented actions or events according to the timeline of the action or the event. In one non-limiting example, the patient may have undergone a treatment for various unrelated medical conditions like brain tumor, chest infection, spinal dislocation and liver cirrhosis. According to an aspect of the disclosure, the segment begin-end detection module (430) may be configured to segment all the actions taken for these medical conditions and show the corresponding medication offered for it. The segment begin-end detection module (430) may generate segments (431, 432, 433, 434, 435) with ambiguous boundaries. Although only five segments (431, 432, 433, 434, 435) have been shown in the present example, it may be understood that there could be more or a smaller number of segments. The segment begin-end detection module (430) correlates the medical condition with the various other parameters including for example the timeline of the medical condition, medication offered against the medical condition. The segment begin-end detection module (430) further creates a visual graph showing the timelines of medications along with the overlaps in the treatment timelines. However, if the specific data related to end of the treatment timeline is not available, or if there are breaks and restarts in the treatment, the boundaries of the graph for such treatment become uncertain and may get mixed with the other graphs. In accordance with an aspect of the disclosure, a hierarchy module (440) may be provided. The hierarchy module (440) may be configured to arrange the segments (441, 442, 443, 444, 445) in order of the timeline. The hierarchy module (440) may be connected to a gap handling module (450) to generate clearly demarcated boundaries. The demarcated segments (441, 442, 443, 444, 445) may be sent to the display (not shown) by the hierarchy module (440) for showing the timeline sequence of the actions or the events related to the patient. The segment begin-end detection module (430) module may be further configured to highlight clinically important information related to each segment. This helps clinician to identify the relevant information from each segment in short time duration as compared to manually finding out the relevant information from different treatments offered over time.

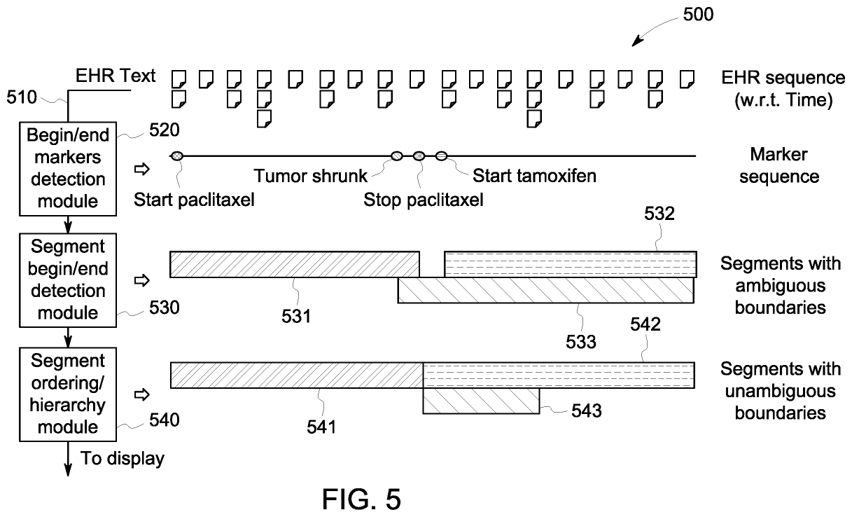

In another non-limiting example, FIG. 5 shows the beginning event or the action may be prescription of a drug. A color-coded marker sequence may be generated corresponding to various electronic health records (EHRs) by the begin-end marker detection module. The EHRs may be displayed as a text (510). Further, the patient may have been prescribed to undergo chemotherapy treatment using a drug Paclitaxel. This marks the beginning of Chemotherapy Action. After a few days of this treatment, a Computed Tomography (CT) Scan may indicate that the tumor has shrunk in size. Upon seeing this result, the doctor may decide to stop the drug Paclitaxel and on a future date and start the patient on another drug Tamoxifen. This marks the end of Chemotherapy Action and beginning of Response Event and Hormonal Therapy Event. The prescription information of the patient in past may be time stamped and stored in the computer memory or any other suitable electronic storage medium.

The begin-end marker detection module (520) may be configured to access the scanning information from the electronic storage medium. From the information stored on the electronic storage medium, the begin-end marker detection module (520) may be further configured to identify the prescribed drug including the date of administration of the first dose. The begin-end marker detection module (520) may be configured to identify all such actions or events associated with the patient over the entire duration of the treatment. In one aspect of the disclosure, the begin-end marker detection module (520) may be configured to read the text information stored in the electronic storage medium and convert the information into a user viewable graph. Further, the segment begin-end detection module (530) may be configured to segment the Chemotherapy Action, Hormonal Therapy Action and the Response Event. However, the exact dates of when Paclitaxel was stopped or when Tamoxifen was started may not be available in EMRs and hence the segment begin-end detection module (530) may generate segments (531, 532, 533) with ambiguous boundaries. Although only three segments (531, 532, 533) have been shown in the present example, it may be understood that there could be more or a smaller number of segments.

The segment begin-end detection module (530) correlates the prescribed drugs with the various other parameters including for example the medical condition, affected anatomical region etc. The segment begin-end detection module (530) further creates a visual graph showing the timelines of medications along with the overlaps in the treatment timelines. However, if the specific data related to end of the treatment timeline is not available, or if there are breaks and restarts in the treatment, the boundaries of the graph for such treatment become uncertain and may get mixed with the other graphs. In accordance with an aspect of the disclosure, a hierarchy module (540) may be provided. The hierarchy module (540) may be configured to arrange the segments (541, 542, 543) in order of the timeline. The hierarchy module (540) may be connected to a gap handling module (550) to generate clearly demarcated boundaries. The demarcated segments (541, 542, 543) may be sent to the display (not shown) by the hierarchy module (540) for showing the timeline sequence of the actions or the events related to the patient. The segment begin-end detection module (530) module may be further configured to highlight clinically important information related to each segment. This helps clinician to identify the relevant information from each segment in short time duration as compared to manually finding out the relevant information from different treatments offered over time.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

We claim:

1. A method for patient history analysis and time-line segmentation, the method comprising:
acquiring health records of a patient using a plurality of medical devices over duration of a treatment, wherein health records comprise at least one action or an event related to the patient;
identifying beginning and end of the at least one action or the event related to the patient and marking beginning and end boundary of the at least one action or the event;
segmenting the at least one action or the events related to the patient by generating ambiguous timeline boundaries of the at least one action or the event; and
demarcating the segment boundaries and arranging the segments in order of timeline.

2. The method as claimed in claim 1, wherein acquiring health records of the patient using the plurality of medical devices over a period of time further comprises storing the health records in a computer readable media.

3. The method as claimed in claim 1, wherein demarcating the segment boundaries and arranging the segments in order of timeline further comprises displaying the segmented health records over a display device.

4. The method as claimed in claim 3, wherein displaying the segmented health records over the display device further comprises highlighting at least one of the clinically important action or the event related to the patient from the segmented health records.

5. The method as claimed in claim 1, wherein the at least one action comprises beginning or end of a treatment, a therapy and a medication, and the event comprises admission or discharge of the patient from the hospital, medical imaging, and testing of patient body parameters.

6. The method as claimed in claim 1, wherein the begin-end marker detection module is configured to read text information stored in the electronic storage medium and convert text information into a user viewable graph for displaying over the display device.

7. The method as claimed in claim 1, wherein demarcating the segment boundaries and arranging the segments in order of timeline comprises using a gap handling module connected to demarcate the segments and a hierarchy module configured arrange the demarcated boundaries.

8. The method as claimed in claim 1, wherein segmenting comprises identifying the timeline boundaries of the at least one action or the events and arranging the at least one segmented action or the event according to the timeline of the at least one action or the event.

9. A system for patient history analysis and time-line segmentation, the system comprising:
a health record system configured to store a plurality of health records of a patient acquired over a duration of a treatment, wherein health records comprise at least one action or an event related to the patient;
a begin-end marker detection module connected to the health record system to retrieve the health records of the patient, wherein the begin-end marker detection module is configured to identify beginning and end of at least one action or the event related to the patient and mark the beginning and end boundary of the at least one action or the event;
a segment begin-end detection module configured to segment the at least one action or the event related to the patient by generating ambiguous timeline boundaries of the at least one action or the event; and
a gap handling configured to demarcate the segment boundaries; and
a hierarchy module configured arrange the segments in order of timeline.

10. The system as claimed in claim 9, wherein the hearth record system comprises electronic health record system.

11. The system as claimed in claim 9, wherein the health record system comprises a computer memory, a sever or any other electronic media configured to store the health records.

12. The system as claimed in claim 9, further comprising a display device connected to the hierarchy module and configured to display the demarcated segment boundaries arranged in order of timeline.

13. The system as claimed in claim 12, wherein displaying demarcated segment boundaries arranged in order of timeline over the display device further comprises highlighting at least one of the clinically important action or the event related to the patient from the segmented health records.

14. The system as claimed in claim 9, wherein the at least one action comprises beginning or end of a treatment, a therapy or a medication and the event comprises admission or discharge of the patient from the hospital, medical imaging, and testing of patient body parameters.

15. The system as claimed in claim 9, wherein the begin-end marker detection module is configured to read text information stored in the electronic storage medium and convert text information into a user viewable graph for displaying over the display device.

16. The system as claimed in claim 9, wherein segmenting comprises identifying the timeline boundaries of the at least one action or the event and arranging the at least one segmented action or the event according to the timeline of the at least one action or the event.
